(19) United States
(12) Patent Application Publication
(10) Pub. No.: US 2019/0237178 A1
(43) Pub. Date: Aug. 1, 2019

(54) METHOD TO REDUCE ERRORS, IDENTIFY DRUG INTERACTIONS, IMPROVE EFFICIENCY, AND IMPROVE SAFETY IN DRUG DELIVERY SYSTEMS

(71) Applicant: Norman Shaye, Lakewood, NJ (US)

(72) Inventor: Norman Shaye, Lakewood, NJ (US)

(21) Appl. No.: 15/883,075

(22) Filed: Jan. 29, 2018

(51) Int. Cl. G16H 20/13; G16H 10/65

(52) U.S. Cl. CPC G16H 20/13; G16H 10/65

(57) ABSTRACT

This embodiment reduces the chances of interactions between Prescriptions (Rx's), received from medical professionals, and over-the counter vitamins, minerals and/or supplements. These interactions could reduce the effectiveness, and/or cause dangerous reactions. The interaction could provide guidance of when to take, not to take or how to take the Prescriptions (Rx's) and/or over-the counter vitamins, minerals and/or supplements. This embodiment also reduces the interaction to the patient's existing Rx's and previous Rx's.

If drug interactions are identified the onsite pharmacist will be notified. The pharmacist will review drug interactions, if any, with the Rx (dosage, frequency of taking drug, etc.) and over-the-counter drugs, and discuss with the customer, if necessary.

### BACKGROUND OF THE INVENTION

Several years ago the AMA's New England Journal of Medicine estimated that each year approximately 80,000 deaths occur in the USA due to medical errors. Some of these errors occur when prescriptions (Rx) are filled at a drug store and/or when over the counter drugs (example: vitamins, minerals, herbals, supplements, painkillers etc.) interact with each other and prescriptions.

Currently interactions of various Rx drugs depend on the knowledge and memory of the pharmacists and the literature provided to the customer at checkout. Information on the interactions of customer's various Rx's with over-the-counter drugs and with over-the-counter drugs themselves is are not provided. The customers have to determine, for themselves, if an interaction will occur using the Internet and/or literature (example: AARP magazine).

With the introduction of having electronic prescriptions sent from a Doctor directly to drug dispensing businesses (Example: CVS, Walgreen, etc.) the need for an individual to read the Rx will be eliminated. Having someone read the Rx (from a paper Rx or a printout), possibly someone else to obtain the correct drug with the correct strength from the shelves, and then a third person to count the correct number of drugs introduces the chances of mistakes. Even though the personnel working at drug dispensing stores are from highly trained licensed pharmacist to trained clerks, errors can still occur.

The time Doctors spend with patients are decreasing due to reduced reimbursement from various medical plans. Because of this, the chances of mistakes with the Rx not identifying drug interactions (both from Rx's and over-the-counter could increase identify drug interactions and therefore improved customer safety in drug delivery systems.

With the availability of some new technologies such as, “Smart Phones”, Barcodes and Barcode readers; and Radio Frequency IDs (RFIDs), advances in the ease of entering over-the-counter purchases will help increase awareness of drug interactions.

14 claims, 6 Drawing Sheets

### BRIEF SUMMARY OF THE INVENTION

This embodiment identifies possible drug interactions with Rx's and over-the-counter drugs.

This embodiment will improve the method of determining what prescription Rx was ordered and what over the counter drugs are being purchased. This will be accomplished by scanning the bar codes of over-the-counter vitamins, minerals and supplements). The bar codes will be scanned using a laser scanner or a “smart phone”. This information will be sent to the local (or regional) computer to determine the Rx and over-the-counter drug types. The possible interaction with their prescription drugs and the over-the counter vitamins, minerals and/or supplements drugs can now be determined from a drug interaction (both Rx's and over-the-counter) data base. Also, this will help detect if the correct Rx drug matches the prescription.

### BRIEF DESCRIPTION OF THE DRAWINGS

FIGS. 1, 2, 3, and 4 Canceled

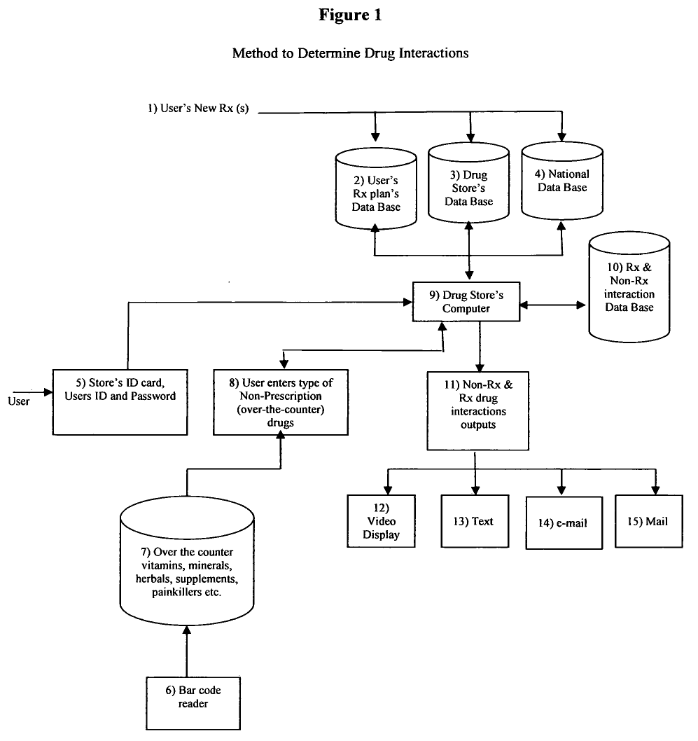

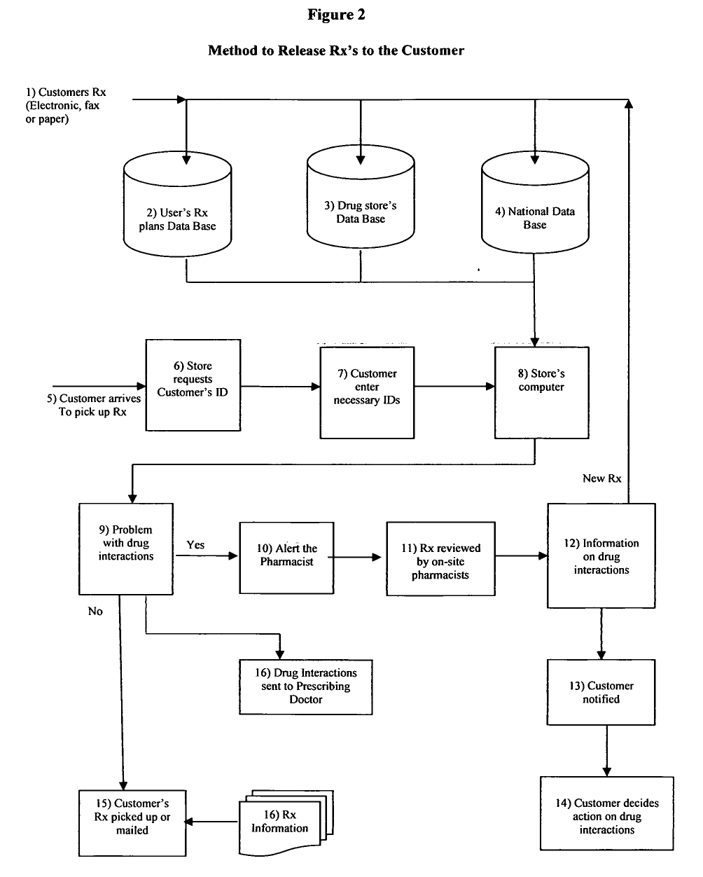

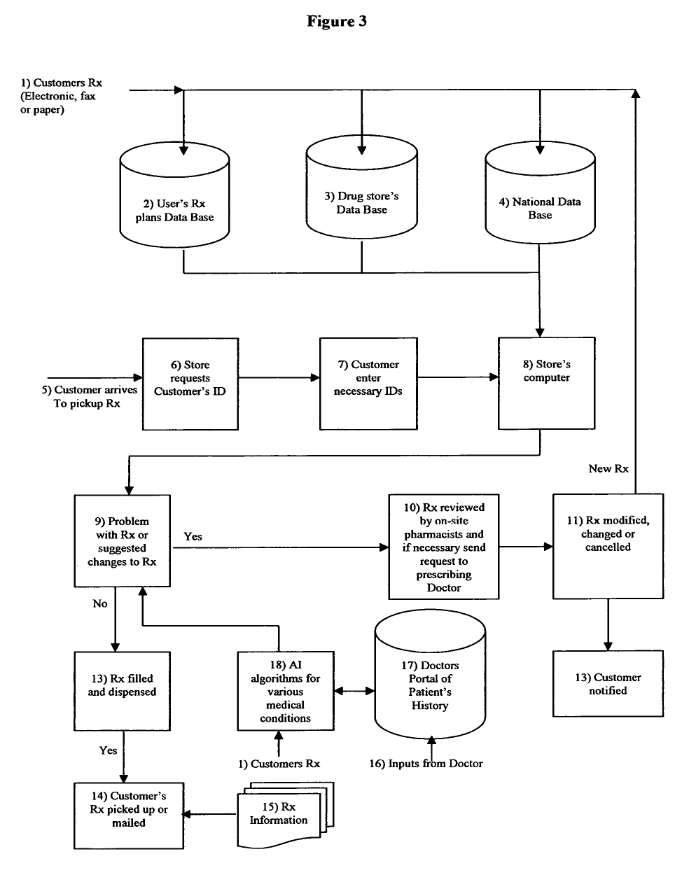

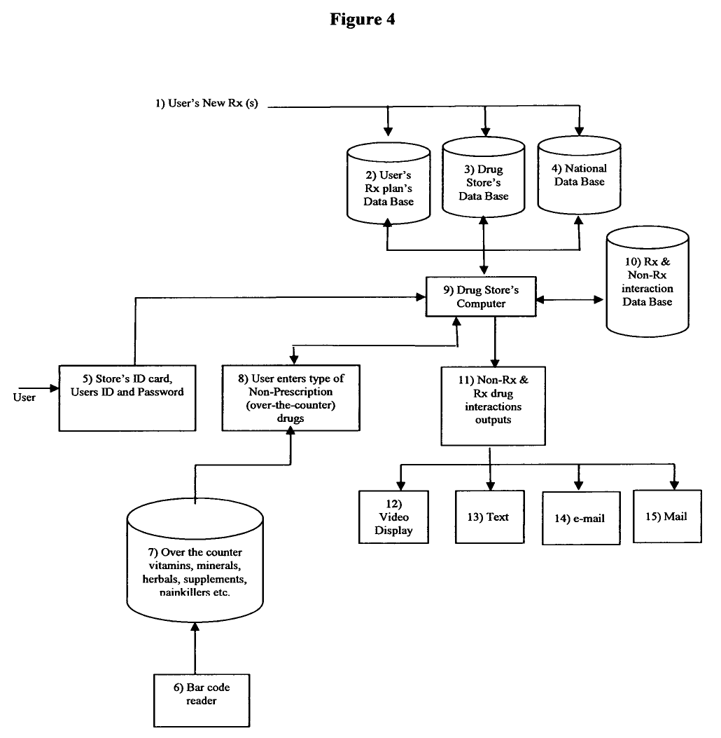

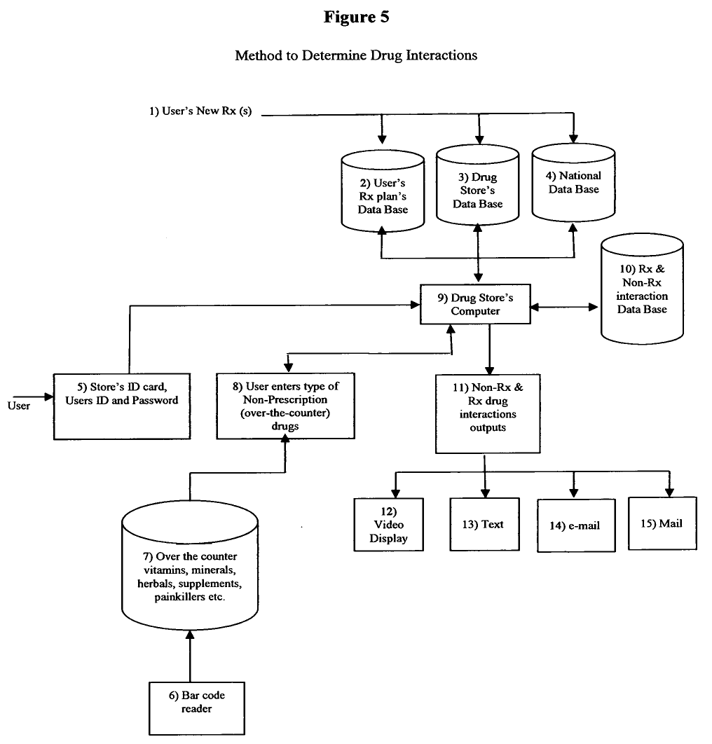

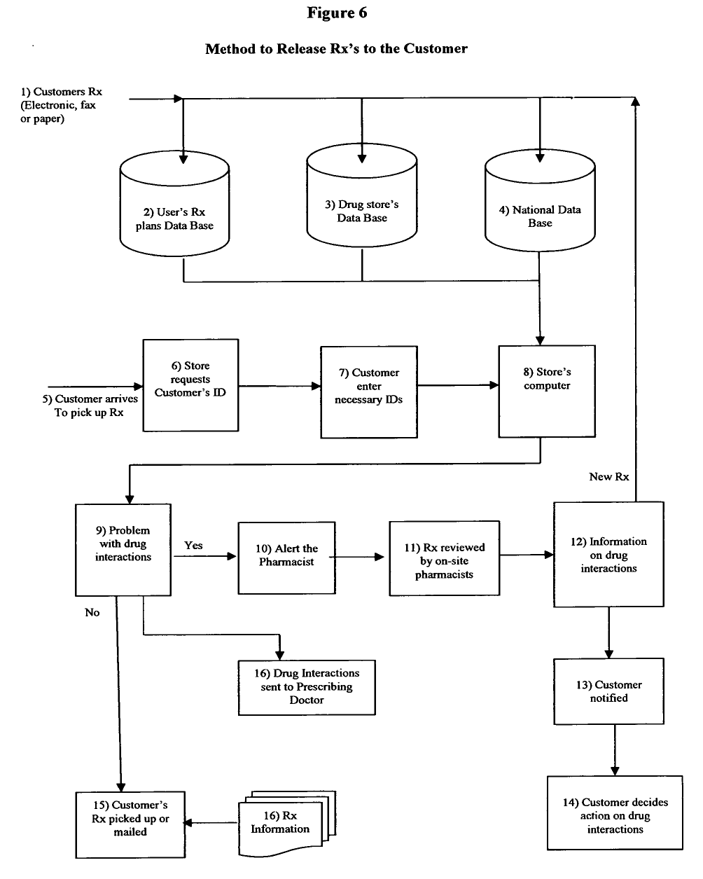

FIG. 5 shows an exemplary block diagram of the process of identifying interaction of the Rx with other Rx the customer is currently taking and with over-the-counter various vitamins, minerals and supplements. This consists of ensuring that the delivered Rx does not cause unnecessary and possible dangerous interactions. The components in the Figure are:
1. User enters new Rx (could be paper or electronic).
2. User's Rx plan database
3. Drug store's database.
4. National database.
5. User enters store's ID, user ID and password. This information is sent to the drug store's computer (number 9).
6. Using a “smart phone” or bar code reader users obtains the type of over-the-counter various vitamins, minerals and supplements.
7. A database containing the information from the “smart phone” or bar code is accessed and this is sent to number 8.
8. The type of non-prescription (over-the-counter drugs) are determined and sent to drug store's computer (number 9).
9. Data from the various inputs above are sent to the store's computer (located locally or central).
10. A database containing Rx and non-Rx interactions are accessed by the store's computers.
11. Any interactions from the Rx and non-Rx drugs are summarized.
12. 13. 14. &15. Various outputs are used to relay the interaction to the customer depending on their preference. This was determined when the customer signed up for this service,

FIG. 6 shows an exemplary block diagram of the process of releasing the Rx to the customer. The components in the Figure are:
1. Customer's Rx is received (see FIG. 5).
2. Databases from the user's Rx plan. Contains the customers Rx history, and over-the-counter drugs
3. The Drug's stores Database. Also contains the customers Rx history and/or over-the-counter drugs.
4. National Database. Also contains the customers Rx history. This is needed if the customer uses several drug delivery stores.
5. Customer arrives to pick up Rx.
6. Store requests customer IDs.
7. Customer enters necessary IDs.
8. Information stored in the store's computer.
9. Is a problems of drug interactions identified with the Rx (see FIG. 5)?
10. If yes, the pharmacist is alerted.
11. Rx reviewed by on-site pharmacist
12. Information of possible drug interactions sent to the various databases (numbers 2, 3 and 4 above).
13. Customer notified of possible drug interactions with the Rx.
14. Customer decides if they want to obtain the Rx and/or purchase some of the over-the-counter drugs, and/or contact the Doctor, or all/some of the options.
15. Customer picks up Rx or Rx is mailed.
16. Rx information printed (such as interactions) and delivered with the Rx.
17. Drug interactions with Rx, if any, could be sent to the prescribing Doctor.

### DETAILED DESCRIPTION OF THE INVENTION

This embodiment identifies possible drug interactions with Rx's and over-the-counter drugs.

The embodiment will identify interactions between various over-the-counter vitamins, minerals and supplements; and with the patient prescription drugs. To identify interaction between various prescription drugs the patient is currently taking will occur by accessing databases that contains the history of the patient's drugs. These could be one of several databases such as 1) the user's Rx plan, 2) the drug stores and/or, 3) a national database. These databases could be local or centrally located. The types of Rx's used by an individual will be stored in one or more databases. The bar code of over-the-counter vitamins, minerals and supplements will be laser scanned by the user (patient). An example of a laser scanner is the type used at check out counters at super markets. For “smart phone” users an app will be provided by the pharmacy that can read the bar code or other markings. Individuals using the system will input the information using the store's ID card 8 and then their User Name and PIN. The results of interactions, if any, will be displayed on the “smart phone's screen” or on a flat screen display. As an option the interactions can be sent to the user as an email, FAX, printed and/or mailed.

This embodiment reduces the chances of interactions between Prescriptions (Rx's) received from medical professionals and with over-the counter vitamins, minerals and/or supplements. These interactions could reduce the effectiveness, and/or cause dangerous reactions. The interaction could provide guidance of when to take, not to take or how to take the Prescriptions (Rx's) and/or over-the counter vitamins, minerals and/or supplements. This embodiment also reduces the interaction to the patient's existing Rx's and previous Rx's problems.

Although the above descriptions may contain specific details, they should not be construed as limiting the claims in any way. Based on the teachings herein, various modifications and changes may be made thereto by those skilled in the art and therefore fall within the spirit and scope of the embodiment, as defined by the appended claims.

The claimed invention is:

1. (canceled)

2. (canceled)

3. (canceled)

4. (canceled)

5. (canceled)

6. (canceled)

7. (canceled)

8. (canceled)

9. (canceled)

10. (canceled)

11. A method to improve patient safety by reducing possible drug interactions by determining, using electric identifying systems, such as bar codes and RFIDs, what prescription Rx's were ordered, what prescription Rx's were filled and what over the counter drugs were purchased.

12. The method in claim 11 will determine if a drug interaction is indicated and an alert will be given to the on-site pharmacist and/or the customer/patient.

13. The method in claim 11 will use patient's information stored in onsite, regional and/or national databases.

14. The method in claim 11 will use various scanning devices (using RFIDs or bar codes, as examples) to identify over-the-counter drug products (such as vitamins, minerals and/or supplements).
